(19) United States
(12) Patent Application Publication
(10) Pub. No.: US 2012/0283642 A1
(43) Pub. Date: Nov. 8, 2012

(54) MICRONEEDLE DEVICE

(75) Inventors: Toshiyuki MATSUDO, Tsukuba-shi (JP); Tetsuji KUWAHARA, Tsukuba-shi (JP); Seiji TOKUMOTO, Tsukuba-shi (JP)

(73) Assignee: HISAMITSU PHARMACEUTICAL CO., INC., Tosu-shi (JP)

(21) Appl. No.: 13/460,629

(22) Filed: Apr. 30, 2012

Related U.S. Application Data
(63) Continuation of application No. 12/738,123, filed on Apr. 15, 2010, now abandoned.

(30) Foreign Application Priority Data
Oct. 18, 2007 (JP) 2007-271732

(51) Int. Cl. A61M 5/00

(52) U.S. Cl. 604/173

(57) ABSTRACT

A microneedle device having a coating including a high molecular weight pharmaceutical compound substantially uniformly is provided. A microneedle device (1) includes a plurality of microneedles (3) on a microneedle substrate (2), which are capable of piercing the skin. A portion of or entire surface of the microneedles (3) and/or the microneedle substrate (2) has a coating including a coating carrier in a solid state. The coating carrier includes a high molecular weight pharmaceutical compound and polysaccharides compatible with the high molecular weight pharmaceutical compound. Herein, as the polysaccharide, for example, pullulan or hydroxypropylcellulose can be used.

### CROSS-REFERENCE TO RELATED APPLICATIONS

This is a Rule 53(b) Continuation of application Ser. No. 12/738,123 filed Apr. 15, 2010, which is a 371 of PCT Application No. PCT/JP2008/068685 filed Oct. 15, 2008, which claims benefit to Japanese Patent Application No. 2007-271732 filed Oct. 18, 2007. The above-noted applications are incorporated herein by reference in their entirety.

### TECHNICAL FIELD

The present invention relates to a microneedle device having a plurality of microneedles on a substrate, which are capable of piercing the skin, for administering a drug through the skin.

### BACKGROUND ART

A microneedle device has been conventionally known as a device for enhancing transdermal absorption of drugs. Microneedles provided on the microneedle device are intended to pierce the stratum corneum that is the outermost layer of the skin and have been proposed in various sizes and shapes, and thus the microneedle device is expected to provide a non-invasive administration method (for example, Patent Document 1).

Furthermore, a variety of drug administration methods using a microneedle device have been proposed. An example of such methods includes coating a drug on a surface of a microneedle, providing a microneedle with a groove or a hollow part through which a drug or a body composition is allowed to pass, mixing a drug with a microneedle itself, and the like. It is reported that a preferable reservoir medium contains a saccharide and, in particular, a stabilizing saccharide such as lactose, raffinose, trehalose, or sucrose, which forms glass (noncrystalline solid material) (Patent Document 2).

One of the methods for efficiently promoting transdermal absorption of drugs by using a microneedle device is a method of coating a drug on a part of the surface of a microneedle device. Specifically, when a drug is coated on a part of the microneedle device (in particular, only a microneedle), all or almost all of the applied drugs are delivered into the body. Thus, the microneedle device is useful as extremely efficient and accurate administration means. Any of such proposed microneedle devices have extremely small protrusions having the height of about several tens to several hundreds micrometers. Therefore, it can be easily assumed that the transdermal absorption and efficiency of drugs are significantly different depending upon drug application methods.

Patent Documents 3 to 5 disclose that a coating carrier on a microprotrusion array used in transdermal administration of a vaccine or the like is made of a biocompatible carrier selected from human albumin, polyglutamic acid, polyaspartic acid, polyhistidine, pentosan polysulfate, and polyamino acid, as well as a reducing sugar, a non-reducing sugar and polysaccharide.

Furthermore, Patent Document 6 discloses that an example of the main component of the substrate or the protruding part includes a biodegradable polymer such as polylactic acid, saccharides such as glucose, maltose, fructose, and pullulan. However, since the tip of the protruding part has a flat shape or a round shape, the protruding part cannot penetrate the stratum corneum but can only stretch the epidermis. Therefore, penetration of the protruding part into the stratum corneum and mixing with the body fluid are not considered. In addition, the biodegradable polymer is used as the main component of the substrate or protrusion and a drug is contained therein, or a drug is coated on the protrusion by using a solvent, thus posing problems from the viewpoint of absorption efficiency.
Patent Document 1: National Publication of International Patent Application No. 2001-506904
Patent Document 2: National Publication of International Patent Application No. 2004-504120
Patent Document 3: National Publication of International Patent Application No. 2004-528900
Patent Document 4: National Publication of International Patent Application No. 2007-501070
Patent Document 5: National Publication of International Patent Application No 2007-501071
Patent Document 6: Japanese Patent Application Unexamined Publication No. 2007-089792

### DISCLOSURE OF THE INVENTION

### Problems to be Solved by the Invention

It is reported that in order to coat a desired amount of pharmaceutical compound (a low molecular weight compound and a high molecular weight agent such as peptide and protein) to the tip portion of a microneedle, it is effective to add a pharmaceutical compound carrier (thickener) on a coating solution. It is reported that a water-soluble polymer such as PVA actually enables an efficient drug delivery (WO2007-091608). However, in the case where the pharmaceutical compound is limited to high molecular weight pharmaceutical compounds such as peptide and protein, mixing of most of water soluble polymers to be used as a carrier with the high molecular weight pharmaceutical compounds (peptide, protein, and the like) causes an aggregation phenomenon or a phase separation phenomenon. Thus, it is difficult to obtain a uniform coating solution. Unless the coating solution contains a high molecular weight pharmaceutical compound uniformly, it is not possible to accurately control the amount of the high molecular weight pharmaceutical compound to be coated on the microneedles.

Therefore, an object of the present invention is to provide a microneedle device having a coating that includes a high molecular weight pharmaceutical compound substantially uniformly.

### Means for Solving the Problems

In order to achieve the above-mentioned object, the present inventors have keenly studied and carried out screening of pharmaceutical compound carriers. As a result, they have found that the use of some water soluble polysaccharides enables reliable uniform mixing of high molecular weight pharmaceutical compounds without causing an aggregation phenomenon or a phase separation phenomenon, and reached the present invention.

The present invention relates to a microneedle device including a plurality of microneedles on a substrate, which are capable of piercing the skin, wherein a portion of or entire surface of the microneedles and/or the substrate has a coating including a coating carrier in a solid state, the coating carrier including a high molecular weight pharmaceutical compound and a polysaccharide compatible with the high molecular weight pharmaceutical compound. Herein, an example of the polysaccharide includes one or two or more selected from the group consisting of pullulan, hydroxypropylcellulose, and hyaluronic acid.

### Advantages of the Invention

According to the present invention, it is possible to obtain a microneedle device having a coating that contains a high molecular weight pharmaceutical compound substantially uniformly. That is to say, a coating carrier used in the present invention includes a high molecular weight pharmaceutical compound and a polysaccharide compatible with the high molecular weight pharmaceutical compound. Thus, a coating solution that is a viscous aqueous solution including a high molecular weight pharmaceutical compound substantially uniformly can be obtained, and aggregation or phase separation of high molecular weight pharmaceutical compound due to the addition of a water soluble polymer can be suppressed. Since the solution is substantially uniform, the solution can be coated on the microneedle with high accuracy. The coating amount of the high molecular weight pharmaceutical compound can be controlled by adjusting the viscosity of the water soluble polymer. Thus, usability of the microneedle can be specifically enhanced.

### BRIEF DESCRIPTION OF THE DRAWINGS

FIG. 1 shows a microneedle device in accordance with one Example of the present invention: (a) is a perspective view thereof, and (b) is a sectional view of (a) taken along line A-B;

FIGS. 2(a) to 2(c) show an example of a method of coating microneedles;

FIG. 3 is a graph showing an example of a change of weight over time after various types of polymer aqueous solutions are spread;

FIG. 4 is a graph showing an example of a measurement result of BSA content for each pullulan concentration; and

FIG. 5 is a graph showing an example of a correlation between the pullulan concentration and the viscosity.

### DESCRIPTION OF SYMBOLS

1, 22 microneedle device
2 microneedle substrate
3, 21 microneedle
4, 40 coating
23 table
24 aperture
25 mask plate
27 coating solution
28 spatula

### BEST MODE FOR CARRYING OUT THE INVENTION

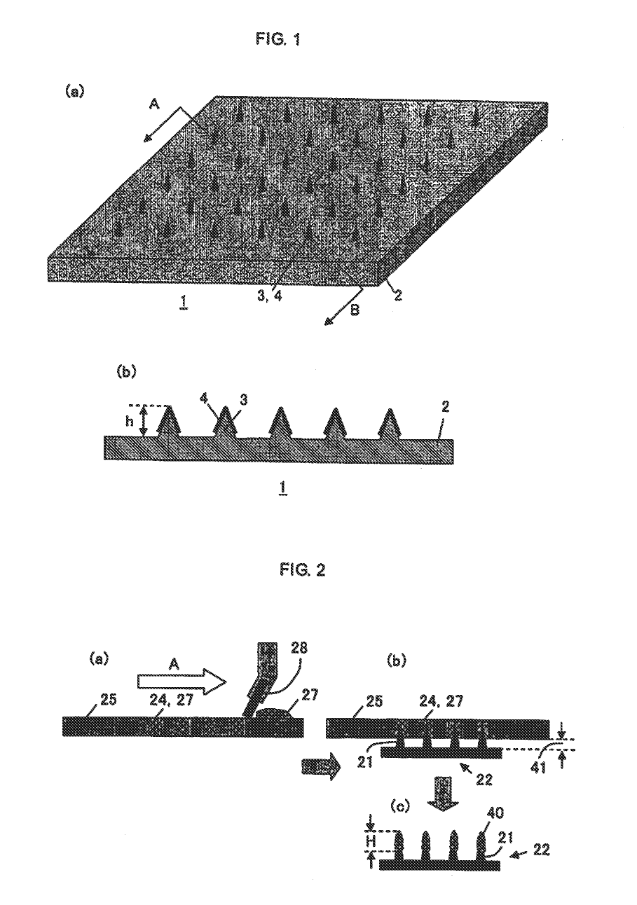

FIG. 1 shows a microneedle device in accordance with one Example of the present invention: (a) is a perspective view thereof, and (b) is a sectional view of (a) taken along line A-B. As shown in FIG. 1(a), a microneedle device 1 includes a microneedle substrate 2, and a plurality of microneedles 3 that are capable of piercing the skin and are arranged in a two-dimensional array on the microneedle substrate 2. On the microneedles 3, a coating 4 is provided by using a coating carrier as means for holding a high molecular weight pharmaceutical compound.

The coating 4 is formed by fixedly attaching a coating solution containing a high molecular weight pharmaceutical compound and a polysaccharide compatible with the high molecular weight pharmaceutical compound to a portion of or entire surface of the microneedles 3 and/or the microneedle substrate 2. Herein, the “high molecular weight pharmaceutical compound” denotes a pharmaceutical compound having a molecular weight of 1000 or more. The term “compatible with” is defined as being in a state, in a range of a visual evaluation, which is free from phase separation and aggregation in the centrifugal operation after a solution is adjusted. The polysaccharide compatible with the high molecular weight pharmaceutical compound may include, for example, the below-mentioned pullulan, hydroxypropylcellulose, hyaluronic acid, and the like. The term “solid” denotes that a coating solution keeps a state in which subjects are uniformly deposited. Immediately after the coating solution is coated, the coating solution is solidified in a dry state by well-known drying methods such as air drying, vacuum drying, freeze drying, or a combination thereof. However, after transdermal application, water may be contained or an organic solvent may be retained in a state of equilibrium with surrounding atmosphere, so that the coating solution is not necessarily solidified in a dry state.

FIGS. 2(a) to 2(c) show an example of a method for coating microneedles. In this method, firstly, as shown in FIG. 2(a), a coating solution 27 is swept on a mask plate 25 by a spatula 28 in a direction shown by an arrow A so as to fill the coating solution in apertures 24. Then, as shown in FIG. 2(b), microneedles 21 are inserted into the apertures 24 of the mask plate 25. Thereafter, as shown in FIG. 2(c), the microneedles 21 are pulled out from the apertures 24 of the mask plate 25. In this way, the microneedles 21 are provided with a coating 40 of a coating solution 27.

The height H of the coating of the microneedle 21 is adjusted by a clearance (gap) 41 shown in FIG. 2(b). The clearance 41 is defined by the length from the base of the microneedle to the surface of the mask (the substrate thickness is not involved), and is set according to the tension of the mask and the length of the microneedle. The length of the clearance 41 preferably ranges from 0 to 500 μm. When the length of the clearance 41 is 0, the entire part of the microneedle 21 is coated.

As mentioned above, a microneedle device includes microneedles (needle parts) that can pierce the skin or mucosa and a microneedle substrate supporting the microneedles. A plurality of the microneedles are arranged on the substrate. A microneedle has a microstructure, and its height (length) h is preferably 50 μm to 500 μm. Herein, the length of the microneedle is set to 50 μm or more for securing the transdermal administration of a pharmaceutical compound, and is set to 500 μm or less for preventing the microneedles from being brought into contact with the nerve, so that the possibility of pain can reliably be reduced and the possibility of bleeding is reliably avoided. Furthermore, when the length is 500 μm or less, it is possible to intradermally administer pharmaceutical compounds efficiently.

Herein, the microneedle is a convex-shaped structure, and it includes a needle structure or a needle-shaped structure in a wide sense. When the microneedle has a conical-shaped structure, the diameter of its base is usually about 50 μm to 200 μm. Furthermore, the shape of the microneedle is not limited to a needle shape having a pointed tip, but may include the shape whose tip is not pointed. The microneedle is preferably made of non-metal synthetic or natural resin materials. Furthermore, the microneedle has a conical shape in this example, but the present invention is not limited to this shape. The shape may be polygonal pyramid such as quadrangular pyramid, or may be other shapes.

The microneedle substrate is a foundation for supporting the microneedles, and there are no particular limitations on its shape. For example, the microneedle substrate may be a substrate provided with through holes, which makes it possible to administer a pharmaceutical compound from the rear surface of the substrate. The material of the microneedles or the substrate may be silicon, silicon dioxide, ceramics, metals (stainless steel, titanium, nickel, molybdenum, chromium, cobalt, and the like), and synthetic or natural resin materials, and the like. However, when the antigenicity of the microneedle and the cost of the material are taken into consideration, the synthetic or natural resin materials including a biodegradable polymer such as polylactic acid, polyglycolide, polylactic acid-co-polyglycolide, pullulan, capronolactone, polyurethane, and polyanhydride, or a non-degradable polymer such as polycarbonate, polymethacrylic acid, ethylenevinylacetate, polytetrafluoroethylene, polyoxymethylene, or the like are particularly preferred. Furthermore, polysaccharides such as hyaluronic acid, pullulan, dextran, dextrin, or chondroitin sulfate are also preferred.

The space between rows is provided to give the density of the microneedles (needle parts) typically at about 1 to 10 per 1 millimeter (mm) in a row of the needles. Generally, the rows are spaced at substantially equal intervals to the space of the needles aligned in the row, and have the density of 100 to 10000 needles per 1 cm². When the density is 100 needles or more, piercing the skin can be efficiently carried out. The density of more than 10000 needles makes it difficult to provide the microneedles with the strength capable of piercing the skin.

Examples of a method of manufacturing the microneedles include wet etching processing or dry etching processing using a silicon substrate, precision machining using metal or resin (such as discharge machining, laser machining, dicing processing, hot embossing, and injection molding), mechanical cutting, and the like. With such processing methods, the needle part and the support part are molded into one unit. An example of methods for hollowing the needle part includes a method of performing secondary processing by using laser machining and the like after the needle part is prepared.

When microneedles are coated, in order to minimize the change in the concentration and the physical properties of drugs due to the volatilization of a solvent in a coating solution, it is preferable that the temperature and humidity of the environment in which a device is installed be controlled to be constant. In order to prevent the transpiration of the solvent, it is preferable to control either or both of the reduction in the temperature and the increase in the humidity. When the temperature is not controlled, the humidity at room temperature is 50 to 100% RH, and preferably 70.0 to 100% RH as the relative humidity. When the humidity is 50% RH or less, the solvent evaporates remarkably, and the change in the physical property of the coating solution occurs. A humidification method is not particularly limited as long as a target humidity state can be secured. An example of the humidification methods includes a vaporization method, a steam method, a water spraying method, and the like. Furthermore, as a thickener to be mixed in the coating solution, a water soluble polymer having a high humidity and moisturizing property so as to suppress the volatility of the solvent as much as possible is preferably selected.

The coating solution can be coated on the microneedles in a state in which the coating solution contains a pharmaceutical compound in purified water and/or a high molecular weight coating carrier. An example of the high molecular weight coating carrier includes polyethylene oxide, polyhydroxymethylcellulose, hydroxypropylcellulose, polyhydroxypropylmethylcellulose, polymethylcellulose, dextran, polyethylene glycol, polyvinyl alcohol, polyvinylpyrrolidone, pullulan, carmellose sodium, chondroitin sulfate, hyaluronic acid, dextran, gum arabic, and the like. The coating solution is coated on a portion or entire or the tip portion of the microneedle, and then dried.

As the coating carrier, polysaccharide carriers with a relative compatibility (property of being mixed homogeneously) with a high molecular weight pharmaceutical compound are preferred. Preferable examples of the polysaccharide carriers include polyhydroxymethylcellulose, hydroxypropylcellulose, polyhydroxypropylmethylcellulose, polymethylcellulose, dextran, polyethylene glycol, pullulan, carmellose sodium, chondroitin sulfate, hyaluronic acid, dextran, and gum arabic. More preferred are hydroxypropylcellulose, pullulan, gum arabic, and the like. Particularly, most preferred are hydroxypropylcellulose (HPC-SSL (molecular weight: 15,000 to 30,000), HPC-SL (molecular weight: 30,000 to 50,000), HPC-L (molecular weight: 55,000 to 70,000), HPC-M (molecular weight: 110,000 to 150,000), HPC-H (molecular weight: 250,000 to 400,000)), pullulan, and hyaluronic acid.

The content of the coating carrier in the coating solution is in the range of 1 to 70% by weight, preferably in the range of 1 to 40% by weight, and particularly preferably in the range of 3 to 25% by weight. Furthermore, the coating carrier may need to have a viscosity to some degree in order to prevent the coating solution from causing a liquid drop. The required viscosity is approximately in the range of 100 to 100000 cps. More preferred viscosity is in the range of 500 to 60000 cps. The viscosity in this range makes it possible to coat a desired amount of coating solution at one time without depending upon the material of the microneedle. Furthermore, in general, as the viscosity becomes higher, the amount of coating solution tends to be increased.

The thickness of the coating on the microneedles is less than 50 μm, preferably less than 25 μm, and more preferably in the range of 1 to 10 μm. Generally, the thickness of the coating is the average thickness measured across the surface of the microneedles after drying. Generally, the thickness of coating can be increased by applying a plurality of coating films of the coating carrier, that is, by repeating the coating process after the coating carrier is solidified.

The height (length) h of the microneedle is preferably 50 μm to 500 μm as mentioned above. The height H of the coating of the microneedle varies depending upon the height h of the microneedle. The height H can be made to be in the range of 0 μm to 500 μm, generally in the range of 10 μm to 500 μm, and preferably in the range of about 30 μm to 300 μm. The coated coating solution is solidified by drying after it is coated.

A liquid composition to be used in coating the microneedles is prepared by mixing a volatile liquid with a biocompatible carrier, a beneficial pharmaceutical compound to be delivered, and any coating auxiliary substances according to circumstances. The volatile liquid can be water, dimethylsulfoxide, dimethylformamide, ethanol, isopropylalcohol and a mixture thereof. The most preferred is water among them. The beneficial pharmaceutical compound in the liquid coating solution or suspension can typically have a concentration in the range of 0.1 to 65% by weight, preferably in the range of 1 to 30% by weight, and more preferably 3 to 20% by weight. The coating is particularly preferred to be in a state of being solidified.

Other well-known formulation auxiliary substances may be added to the coating as long as they do not adversely affect the solubility and viscosity necessary to the coating as well as the properties and physical properties of the dried coating.

The high molecular weight pharmaceutical compound (drug) to be used in the present invention is a high molecular weight compound. The high molecule has a molecular weight of 1000 or more as a guide. The upper limit of the molecular weight is not particularly determined. An example of the high molecular weight compound is thought to include peptide, protein, DNA, RNA, and the like. However, the compounds are not particularly limited, and may include, for example, α-interferon, β-interferon for multiple sclerosis, erythropoietin, follicle stimulating hormone (FSH), follitropin β, follitropin α, G-CSF, GM-CSF, human chorionic gonadotropin hormone, leutinizing hormone, salmon calcitonin, glucagon, GNRH antagonist, insulin, human growth hormone, filgrastin, heparin, low molecular weight heparin, parathyroid hormone (PTH), somatropin, and the like. Furthermore, an example of vaccines may include influenza vaccine, Japanese encephalitis vaccine, rotavirus vaccine, Alzheimer's disease vaccine, arteriosclerosis vaccine, cancer vaccine, nicotine vaccine, diphteria vaccine, tetanus vaccine, pertussis vaccine, Lyme disease vaccine, antirabies vaccine, pneumococcal vaccine, yellow fever vaccine, cholera vaccine, vaccinia vaccine, tuberculosis vaccine, rubella vaccine, measles vaccine, mumps vaccine, botulinum vaccine, herpes virus vaccine, and other DNA vaccine, hepatitis B vaccine, and the like.

Furthermore, pharmaceutical compounds may be vaccine, low molecular weight peptide, saccharide, nucleic acid, and the like, as long as they have a molecular weight of about 1000.

Note here that these drugs may be used singly or in combination of two or more kinds thereof. Drugs in the form of any of inorganic salts and organic salts may naturally be included as long as the salts are pharmaceutically acceptable salts. Furthermore, drugs are basically contained in a coating carrier. However, drugs may not be contained in the coating carrier and can be supplied from through-holes (apertures) provided in the substrate of the microneedles in a subsequent separate step.

### EXAMPLES

### Example 1

### Test to Confirm Compatibility of Various Polymers with BSA and OVA

### Operation Procedure

Mixed aqueous solutions of various polymers and BSA or OVA were prepared according to the conditions shown in Tables 1-1, 1-2 and Tables 2-1, 2-2 below. Compatibility was evaluated by confirming the occurrence of aggregation and the presence of phase separation after centrifugal deaeration (the centrifugation conditions are described in the tables) (homogeneous liquid state marked with O, and heterogeneous liquid states marked with X). In Tables 1-1, 1-2 and Tables 2-1, 2-2, the O mark signifies the ones having compatibility, and the X mark signifies the ones having no compatibility. Note here that the % notation signifies % by weight in the description hereinafter. The measurement of the coating content was performed by measuring BSA or OVA content (deposit amount) after extraction with 1 mL of purified water following the coating by the method described in the above FIG. 2. Furthermore, the term “not available” refers to the fact that no deposition of the polymer on the needles was observed.

TABLE 1-1

| | Polymer | OVA | | Coating |
|---|---|---|---|---|
| | concentration | concentration | Compatibility | content |
| Polymer | (%) | (%) | (centrifugal conditions) | (μg) |
| Pullulan | 20 | 20 | ◯ (15000 rpm × 2 min) | 50 |
| Pullulan | 15 | 16.7 | ◯ (15000 rpm × 2 min) | 16 |
| Pullulan | 7.5 | 16.7 | X | — |
| Pullulan | 5 | 16.7 | X | — |
| Hydroxypropylcellulose-SSL | 15 | 16.7 | X | — |
| Hydroxypropylcellulose-SSL | 20 | 16.7 | X | — |
| Hydroxypropylcellulose-SSL | 25 | 16.7 | X | — |
| Hydroxypropylcellulose-SL | 25 | 25 | ◯ (15000 rpm × 2 min) | 41 |
| Hydroxypropylcellulose-SL | 15 | 30 | X | — |
| Hydroxypropylcellulose-L | 13.3 | 16.7 | ◯ (3000 rpm × 2 min) | 9 |
| Hydroxypropylcellulose-L | 16.7 | 16.7 | ◯ | 16 |
| Hydroxypropylcellulose-L | 20 | 16.7 | X | — |
| Hydroxypropylcellulose-L | 16 | 20 | X | — |
| Hydroxypropylcellulose-L | 13.3 | 20 | X | — |
| Hydroxypropylcellulose-L | 15 | 30 | X | — |
| Hydroxypropylcellulose-H | 4 | 16.7 | X | — |
| Hydroxypropylcellulose-H | 3 | 16.7 | ◯ (5000 rpm × 2 min) | 6 |
| Hydroxypropylcellulose-H | 2 | 16.7 | ◯ (5000 rpm × 2 min) | 5 |
| Hydroxypropylcellulose-H | 1.5 | 16.7 | ◯ (5000 rpm × 2 min) | — |
| Hydroxypropylcellulose-H | 1 | 16.7 | ◯ (5000 rpm × 2 min) | 1 |
| Methylcellulose (SM-25) | 7.5 | 16.7 | X | — |
| Methylcellulose (SM-25) | 4 | 16.7 | X | — |
| Methylcellulose (SM-25) | 2 | 16.7 | X | — |
| Methylcellulose (SM-400) | 5 | 16.7 | X | — |
| Methylcellulose (SM-400) | 4.2 | 16.7 | ◯ (15000 rpm × 2 min) | 17 |
| Methylcellulose (SM-400) | 3 | 16.7 | ◯ (5000 rpm × 2 min) | 7 |
| Methylcellulose (SM-400) | 2.6 | 16.7 | ◯ (15000 rpm × 2 min) | 26 |
| Methylcellulose (SM-400) | 1 | 16.7 | X | — |
| Methylcellulose (SM-8000) | 2.7 | 16.7 | X | — |
| Methylcellulose (SM-8000) | 4 | 16.7 | X | — |
| Methylcellulose (SM-8000) | 3 | 16.7 | X | — |
| Methylcellulose (SM-8000) | 2 | 16.7 | ◯ (15000 rpm × 2 min) | 3 |
| Methylcellulose (SM-8000) | 1 | 16.7 | X | — |

TABLE 1-2

| | Polymer | OVA | | Coating |
|---|---|---|---|---|
| | concentration | concentration | Compatibility | content |
| Polymer | (%) | (%) | (centrifugal conditions) | (μg) |
| Hyaluronic acid (MW900000) | 4 | 30 | ◯ (15000 rpm × 2 min) | 8 |
| Hyaluronic acid (MW900000) | 4 | 16.7 | ◯ (15000 rpm × 2 min) | 2 |
| Hyaluronic acid (MW900000) | 3 | 30 | ◯ (15000 rpm × 2 min) | 13 |
| Hyaluronic acid (MW900000) | 3 | 16.7 | ◯ (15000 rpm × 2 min) | 4 |
| Hyaluronic acid (MW900000) | 2.7 | 16.7 | ◯ (15000 rpm × 2 min) | 12 |
| Hyaluronic acid (MW900000) | 2 | 20 | ◯ (15000 rpm × 2 min) | 9 |
| Hyaluronic acid (MW900000) | 2 | 30 | ◯ (15000 rpm × 2 min) | 16 |
| Hyaluronic acid (MW900000) | 2 | 40 | ◯ (15000 rpm × 2 min) | 24 |
| Hyaluronic acid (MW900000) | 2 | 16.7 | ◯ (15000 rpm × 2 min) | 4 |
| Hyaluronic acid (MW900000) | 1.5 | 16.7 | ◯ (15000 rpm × 2 min) | 7 |
| Hyaluronic acid (MW900000) | 1.5 | 30 | ◯ (15000 rpm × 2 min) | 17 |
| Hyaluronic acid (MW900000) | 1.5 | 40 | ◯ (15000 rpm × 2 min) | 36 |
| Hyaluronic acid (MW900000) | 1.5 | 50 | ◯ (15000 rpm × 2 min) | 83 |
| Hyaluronic acid (MW900000) | 1 | 16.7 | ◯ (15000 rpm × 2 min) | 3 |
| Hyaluronic acid (MW900000) | 1 | 40 | ◯ (15000 rpm × 2 min) | 44 |
| Hyaluronic acid (MW900000) | 1 | 50 | ◯ (15000 rpm × 2 min) | 97 |
| Hyaluronic acid (MW900000) | 0.5 | 40 | ◯ (15000 rpm × 2 min) | 31 |
| Hyaluronic acid (MW900000) | 0.5 | 50 | ◯ (15000 rpm × 2 min) | 73 |
| Hyaluronic acid (MW 2000000) | 2 | 16.7 | X | — |
| Partially neutralized polyacrylate (NP-600) | 3 | 16.7 | ◯ (15000 rpm × 2 min) | Not |
| | | | | available |
| Partially neutralized polyacrylate (NP-600) | 1.5 | 16.7 | ◯ (15000 rpm × 2 min) | Not |
| | | | | available |
| Partially neutralized polyacrylate (NP-800) | 3 | 16.7 | ◯ (15000 rpm × 2 min) | Not |
| | | | | available |
| Partially neutralized polyacrylate (NP-800) | 1.5 | 16.7 | ◯ (15000 rpm × 2 min) | Not |
| | | | | available |
| Polyethylene glycol-20000 | 20 | 16.7 | X | — |
| Polyethylene glycol-20000 | 10 | 16.7 | X | — |
| Polyvinyl alcohol-117 | 10 | 16.7 | X | — |
| Polyvinyl alcohol-117 | 5 | 16.7 | X | — |

TABLE 2-1

| | Polymer | BSA | | Coating |
|---|---|---|---|---|
| | concentration | concentration | | content |
| Polymer | (%) | (%) | Compatibility | (μg) |
| Pullulan | 15 | 30 | ◯ (15000 rpm × 2 min) | 30 |
| Pullulan | 20 | 20 | ◯ (15000 rpm × 2 min) | 18 |
| Hydroxypropylcellulose-SSL | 35 | 16.7 | X | — |
| Hydroxypropylcellulose-SSL | 20 | 16.7 | X | — |
| Hydroxypropylcellulose-SSL | 10 | 16.7 | X | — |
| Hydroxypropylcellulose-SSL | 37.5 | 10 | X | — |
| Hydroxypropylcellulose-SL | 25 | 20 | ◯ (15000 rpm × 2 min) | 20 |
| Hydroxypropylcellulose-SL | 20 | 16.7 | X | — |
| Hydroxypropylcellulose-SL | 16.7 | 16.7 | X | — |
| Hydroxypropylcellulose-L | 15 | 10 | X | — |
| Hydroxypropylcellulose-L | 20 | 16.7 | X | — |
| Hydroxypropylcellulose-L | 16.7 | 16.7 | X | — |
| Hydroxypropylcellulose-L | 13.3 | 16.7 | X | — |
| Hydroxypropylcellulose-M | 5 | 16.7 | X | — |
| Hydroxypropylcellulose-M | 3 | 16.7 | X | — |
| Hydroxypropylcellulose-M | 1 | 16.7 | X | — |
| Hydroxypropylcellulose-H | 3 | 16.7 | X | — |
| Hydroxypropylcellulose-H | 2 | 16.7 | X | — |
| Hydroxypropylcellulose-H | 1 | 16.7 | X | — |
| Methylcellulose (SM-25) | 4 | 16.7 | X | — |
| Methylcellulose (SM-25) | 2 | 16.7 | X | — |
| Methylcellulose (SM-25) | 1 | 16.7 | X | — |
| Methylcellulose (SM-400) | 5 | 16.7 | X | — |
| Methylcellulose (SM-400) | 3 | 16.7 | X | — |
| Methylcellulose (SM-400) | 1 | 16.7 | X | — |
| Methylcellulose (SM-8000) | 3 | 16.7 | X | — |
| Methylcellulose (SM-8000) | 2 | 16.7 | X | — |
| Methylcellulose (SM-8000) | 1 | 16.7 | X | — |
| Dextran (MW-40000) | 50 | 11.4 | X | — |
| Dextran (MW-70000) | 37.5 | 10 | ◯ (15000 rpm × 2 min) | 31 |

TABLE 2-2

| | Polymer | BSA | | Coating |
|---|---|---|---|---|
| | concentration | concentration | | content |
| Polymer | (%) | (%) | Compatibility | (μg) |
| Hyaluronic acid (MW900000) | 3 | 16.7 | ◯ (15000 rpm × 2 min) | — |
| Hyaluronic acid (MW900000) | 3 | 30 | ◯ (15000 rpm × 60 min) | 12 |
| Hyaluronic acid (MW900000) | 2 | 16.7 | ◯ (15000 rpm × 2 min) | — |
| Hyaluronic acid (MW900000) | 2 | 20 | ◯ (15000 rpm × 5 min) | 4 |
| Hyaluronic acid (MW900000) | 2 | 30 | ◯ (15000 rpm × 5 min) | 11.5 |
| Hyaluronic acid (MW900000) | 2 | 40 | ◯ (15000 rpm × 7 min) | 37 |
| Hyaluronic acid (MW900000) | 1 | 16.7 | ◯ (15000 rpm × 2 min) | — |
| Hyaluronic acid (MW900000) | 2.7 | 13.3 | ◯ (15000 rpm × 2 min) | — |
| Partially neutralized polyacrylate (NP-600) | 3 | 16.7 | ◯ (15000 rpm × 2 min) | Not |
| | | | | available |
| Partially neutralized polyacrylate (NP-600) | 1.5 | 16.7 | ◯ (15000 rpm × 2 min) | Not |
| | | | | available |
| Partially neutralized polyacrylate (NP-800) | 3 | 16.7 | ◯ (15000 rpm × ? min) | Not |
| | | | | available |
| Partially neutralized polyacrylate (NP-800) | 1.5 | 16.7 | ◯ (15000 rpm × ? min) | Not |
| | | | | available |
| Hydroxypropylmethylcellulose (90SH-30000) | 3.75 | 10 | X | — |
| Hydroxypropylmethylcellulose (90SH-30000) | 2.5 | 20 | X | — |
| Hydroxypropylmethylcellulose (65SH-1500) | 3.75 | 10 | X | — |
| Hydroxypropylmethylcellulose (65SH-1500) | 2.5 | 20 | X | — |
| Polyvinylpyrrolidone (K29/32) | 35 | 20 | X | — |
| Polyvinylpyrrolidone (K29/32) | 52.5 | 10 | X | — |
| Polyvinylpyrrolidone (K90) | 15 | 20 | X | — |
| Polyvinylpyrrolidone (K90) | 22.5 | 10 | X | — |
| Polyvinylpyrrolidone (K90) | 10 | 13.3 | X | — |
| Polyvinylpyrrolidone (K90) | 24 | 8 | X | — |
| Polyvinylpyrrolidone (K90) | 10 | 26.7 | X | — |
| Polyvinylpyrrolidone (K90) | 6 | 32 | X | — |
| Hydroxypropylmethylcellulose (TC-5) | 15 | 5 | X | — |
| Hydroxypropylmethylcellulose (TC-5) | 10 | 20 | X | — |
| Polyethylene glycol-20000 | 20 | 16.7 | X | — |
| Polyethylene glycol-20000 | 10 | 16.7 | X | — |
| Polyvinyl alcohol 117 | 10 | 16.7 | X | — |
| Polyvinyl alcohol 117 | 5 | 16.7 | X | — |
| Carmellose Na | 7.5 | 10 | X | — |
| Chondroitin sulfate A | 30 | 10 | X | — |

Tables 1-1, 1-2, 2-1 and 2-2 show the results of compatibility of OVA or BSA and each water soluble polymer and the content of BSA or OVA after coating was provided on the microneedles. By optimizing the composition ratio of the pharmaceutical compound to the water soluble polymer, pullulan, hydroxypropylcellulose (HPC), methylcellulose, hyaluronic acid, and polyacrylate Na showed high compatibility. Particularly, pullulan showed high compatibility also with respect to OVA with high concentration. Moreover, when these solutions were used to perform coating on the microneedles by the method described in FIG. 2, pullulan showed the highest value, and hydroxypropylcellulose (SL), methylcellulose, and hyaluronic acid showed higher values in this order, Hydroxypropylcellulose showed a difference in the amount of coating according to its grades. The values showed a tendency to descend in the order of HPC-SL>HPC-L>HPC-H. The reason for this is thought that in hydroxypropylcellulose, the polymer viscoelasticity (viscosity) showed a tendency to rise as the molecular weight was lowered, resulting in the increase of deposition on the microneedles. In addition, methylcellulose showed excellent compatibility with respect to OVA, but did not show excellent conditions with respect to BSA, Hyaluronic acid showed excellent compatibility with respect to both OVA and BSA. Polyacrylate Na showed excellent compatibility, but no deposition on the needles was confirmed, which proved that it was unsuitable as a coating carrier. From the above-mentioned results, by using a coating carrier having a compatibility with a high molecular weight pharmaceutical compound, a coating including a substantially uniform high molecular weight pharmaceutical compound can be achieved.

Note here that the following various polymers were used. Methylcellulose (SM-25, SM-400, and SM-8000) manufactured by Shin-Etsu Chemical Co., Ltd., polyacrylate (NP-600 and NP-800) manufactured by Showa Denko K.K., hydroxypropylmethylcellulose (90SH-30000, 65SH-1500, and TC-5) manufactured by Shin-Etsu Chemical Co., Ltd., and polyvinylpyrrolidone (K29/32 and K90) manufactured by Nippon Shokubai Co., Ltd., were used respectively.

### Example 2

### Test of Dryness of Various Polymer Aqueous Solutions

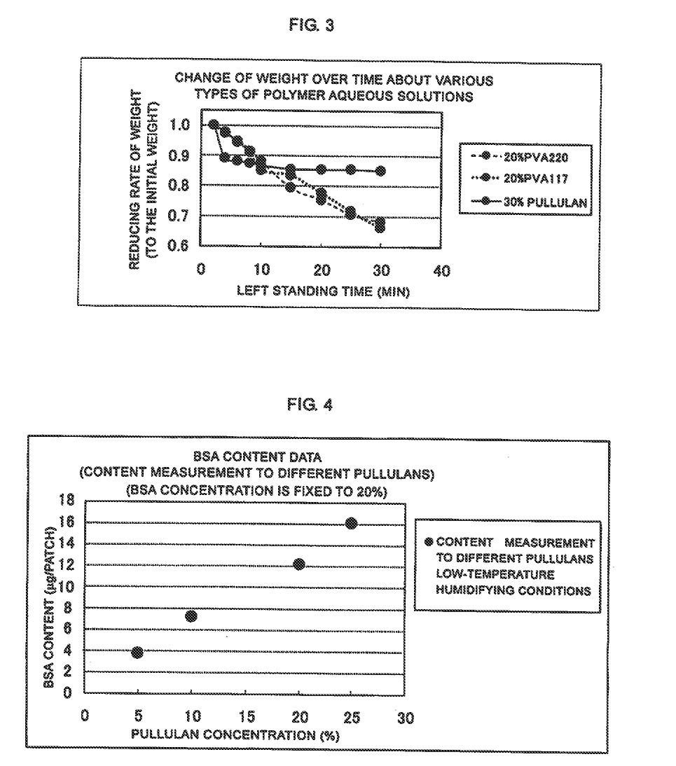

Each of the coating solutions of 20% PVA220, 20% PVA117, and 30% pullulan was spread on a liner to a thickness of 50 μm, and punched out so as to prepare a piece of area of 8 cm², the piece was disposed on an electronic scale and the change of weight over time was measured at room temperature. FIG. 3 is a graph showing an example of a change of weight over time after the above-mentioned various types of polymer aqueous solutions were spread. In FIG. 3, the axis of abscissa shows a time for which the polymer was left standing (min), and the axis of ordinate shows reducing rate of weight (with respect to the initial weight). As shown in FIG. 3, two types of PVAs showed a tendency that the weight was reduced over time during the measurement time, whereas pullulan showed substantially constant weight value although the weight reduction was observed at the initial time. Thus, pullulan showed a stable physical property while it maintained wettability.

### Example 3

### Relation between Pullulan Concentration and Coating Amount of BSA

### Set Condition

### (a) Set Concentration of Coating Solution

pullulan concentration: 5, 10, 20, and 24(%)
BSA (model protein) concentration: fixed to 20(%)

### (b) Microneedle

height: 250 m, 900 needles/cm², formulation area: 1 cm²
   (c) Metal mask plate
pitch: 300 μm, T (mask thickness): 100 μm, aperture part: square shape (200 μm×200 μm)
   (d) Environment: room temperature and low-temperature humidifying conditions

### Operation Procedure

As mentioned above, a coating solution was prepared in which the BSA (bovine serum albumin) concentration was fixed to 20% and the pullulan concentration was set to four concentrations. A coating was carried out by the above-mentioned method shown in FIG. 2. The coating solution was filled in apertures of the metal mask by using a spatula under humidifying condition. Microneedles (needle parts) were inserted into the apertures filled with the coating solution so as to coat the microneedles with the coating solution, and extracted with 1 mL of purified water. Then, the BSA content (deposition amount) was measured by the BCA method (BSA standard) (n=10). Table 3 and FIG. 4 show the results. In FIG. 4, the axis of abscissa shows the pullulan concentration (%), and the axis of ordinate shows the BSA content (μg/patch).

TABLE 3

| Pullulan | BSA content | Coefficient of | Viscosity |
|---|---|---|---|
| concentration (%) | (μg/patch) | variation (CV) (n = 10) | (cps) |
| 5 | 3.7 | 56.1 | 200 |
| 10 | 7.2 | 41.2 | 400 |
| 20 | 12.2 | 25.2 | 2000 |
| 25 | 16.1 | 30.5 | 10000 |

As shown in Table 3, as the pullulan concentration increased, the viscosity of the solution increased. The BSA content was also increased depending upon the increase in viscosity. High viscous solutions (2000 cps and 10000 cps) showed a higher value in the BSA coating amount as compared with the case of low viscous solutions (200 cps and 400 cps), and the coefficient of variation (CV %=(standard deviation/mean value)×100) relating to the BSA content showed a tendency to be reduced. Therefore, the viscosity of the coating solution is preferred to be 500 cps or more from the viewpoint of securing the drug amount to be coated and the accuracy.

### Example 4

### Relation between BSA Concentration in Pullulan and Viscosity

### Set Condition

(a) Set concentration of aqueous solution of pullulan: 5 to 30(%)
(b) Set concentration of pullulan-base coating solution:
pullulan: 10 to 28.5(%)
BSA: 5 to 40(%)

### Operation Procedure

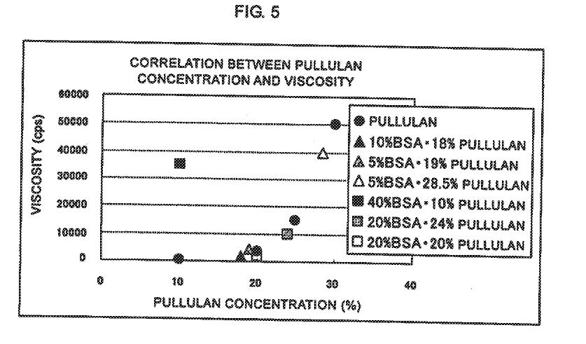

The viscosity of the aqueous solution prepared in the above-mentioned condition was measured by the use of a viscometer (Viscotester VF-04 manufactured by Rion Co., Ltd.). FIG. 5 shows the results as the correlation between the pullulan concentration and the viscosity. As shown in FIG. 5, in the case of an aqueous solution of a single substance of pullulan, as the concentration increased, a quadratic curvilinear increase of the viscosity was confirmed. In the case of a mixture of pullulan and BSA, when the set value of the BSA concentration was low, the viscosity property depending upon the pullulan concentration was confirmed. Under the solution condition in which the BSA concentration was dominant (40% BSA, 10% pullulan), the viscosity deviating from the above-mentioned property depending upon the pullulan concentration was observed. From the results shown in FIG. 5, when the solubility of the high molecular weight pharmaceutical compound with respect to a solvent was low, and when the concentration of the high molecular weight pharmaceutical compound was set to a low value in formulation designing, by appropriately setting the concentration of the coating carrier (for example, pullulan), the viscosity of the coating solution can be controlled and a desired amount of the coating is possible.

### INDUSTRIAL APPLICABILITY

The present invention enables a high molecular weight pharmaceutical compound to be coated on microneedles substantially uniformly. It also enables high accurate coating on microneedles because a solution is uniform. That is to say, since the amount of coating can be controlled by adjusting the viscosity of a water soluble polymer, the usability of the microneedle can be especially enhanced, thus providing the industrial applicability.

1. A microneedle device comprising:
a substrate,
a plurality of microneedles on a substrate, which are capable of piercing a skin,
a coating, which includes a pharmaceutical compound having a molecular weight of 1000 or more, arranged on a portion of or entire surface of the microneedles and/or the substrate,
wherein the microneedles are composed of one material selected from the group consisting of polylactic acid, polyglycolide, polylactic acid-co-polyglycolide, capronolactone, polyurethane, polyanhydride, polycarbonate, polymethacrylic acid, ethylenevinylacetate, polytetrafluoroethylene, and polyoxymethylene, and
wherein the coating includes a polysaccharide as a coating carrier compatible with the pharmaceutical compound, so that the coating contains the pharmaceutical compound substantially uniformly.

2. The microneedle device according to claim 1, wherein the polysaccharide is one or more selected from the group consisting of pullulan, hydroxypropylcellulose, and hyaluronic acid.
